(19) United States
(12) Patent Application Publication
(10) Pub. No.: US 2011/0136166 A1
(43) Pub. Date: Jun. 9, 2011

(54) Imaging Mass Spectrometry for Improved Prostrate Cancer Diagnostics

(75) Inventors: O. John Semmes, Newport News, VA (US); Lisa H. Cazares, Chesapeake, VA (US); Richard R. Drake, Chesapeake, VA (US); Savvas Mendrinos, Virginia Beach, VA (US); Raymond S. Lance, Norfolk, VA (US)

(73) Assignee: Eastern Virginia Medical School, Norfolk, VA (US)

(21) Appl. No.: 12/922,609

(22) PCT Filed: Mar. 13, 2009
(86) PCT No.: PCT/US09/37110

§ 371 Date: Feb. 10, 2011

Related U.S. Application Data
(60) Provisional application No. 61/036,837, filed on Mar. 14, 2008.

(51) Int. Cl. C12Q 1/00

(52) U.S. Cl. 435/40.52

(57) ABSTRACT

The invention provides biomarkers that can discriminate between prostate cancer and normal tissue as well as identification of associated metastatic disease. One biomarker was identified as a peptide fragment of MEKK2. Methods of diagnosing prostate cancer, including metastatic cancer, by detecting the differential expression of one or more biomarkers are also provided.

### RELATED APPLICATION

This application claims the benefit under 35 U.S.C. §119(e) of U.S. Provisional Application No. 61/036,837 filed on Mar. 14, 2008, which is hereby incorporated by reference herein in its entirety.

### STATEMENT REGARDING FEDERALLY SPONSORED RESEARCH

Work described herein was performed with government support under Grant 2 U0 CA085067 awarded by the NIH/NCI Early Detection Research Network. The U.S. Government has certain rights in the invention.

### BACKGROUND OF THE INVENTION

Prostate cancer (PCa) is one of the most common malignancies in the US (1). It is clinically heterogeneous, with a highly variable natural history (2). The discovery and widespread utilization of serum prostate specific antigen (PSA) monitoring for early detection has greatly changed the way prostate cancer is diagnosed and treated. However, PSA lacks specificity as a screening tool for prostate cancer, and there is really no lower limit of PSA that entirely excludes cancer (3). Thus, clinical decision making in PCa places a significant burden upon biopsy results. Ultrasound guided needle biopsy is the standard for diagnosis however, a negative result does not exclude the presence of cancer. Both sampling and analytical variables account for false negative results. In practice, false negative results engender a need for repeat biopsies which can delay diagnosis and treatment or unnecessarily subject cancer-free men to repeat biopsies and their attendant anxiety and risk (4, 5). The heterogeneity of prostate cancer is also a significant problem as the death rates from prostate cancer are relatively low as compared to those from the other major cancers such as lung, pancreas, and colon. The Gleason grading system which has been widely adopted for PCa is a strong predictor of outcome (6). However, a major limitation of this grading system, and a result of aggressive screening procedures, is that a majority of newly diagnosed prostate cancer cases are Gleason grade 6 or 7 tumors. These moderately differentiated tumors can either be indolent or aggressive (7). New methods to assist pathologists in both diagnostic and prognostic decision making are needed to aid in the detection and treatment of prostate cancer.

Matrix-assisted laser desorption/ionization imaging mass spectrometry (MALDI-IMS) of tissue can be used to monitor disease specific alterations in situ at the protein level, both qualitatively and quantitatively (8). This technique holds promise for both the discovery of new molecular markers and the analysis of known markers by examining their expression in tissue and registering output to point of origin within the tissue. MALDI-IMS has the power to link the molecular detail of mass spectrometry with morphology, generating mass spectra correlated to orthogonally characterized locations within a tissue section (9-11). Several recent studies underscore the potential of MALDI-IMS for clinical histopathology applications. Protein expression profiles obtained from tissue could discriminate lung cancer subtype (12). Tumor histology, therapeutic response, and patient survival was shown to correlate with the protein expression patterns obtained from direct tissue analysis in breast tumors (13, 14). Protein expression patterns and images were also found to correlate with brain tumor histology and patient survival (15). Similar discovery efforts using MALDI-IMS have yielded potential candidates in ovarian, colon and prostate cancer (16, 18).

Unless otherwise defined, all technical and scientific terms used herein have the same meaning as commonly understood by one of ordinary skill in the art to which this disclosure belongs. Although methods and materials similar or equivalent to those described herein can be used in the practice or testing of the present disclosure, suitable methods and materials are described below. All publications, patent applications, patents, and other references mentioned herein are incorporated by reference in their entirety. In addition, the materials, methods, and examples are illustrative only and not intended to be limiting.

### BRIEF SUMMARY OF THE INVENTION

The invention is directed to detecting and determining prostate health by proteomic profiling. The invention provides biomarkers that have been profiled and characterized from clinical samples such as prostatic tissue. These biomarkers may be used to develop proteomic profiling systems for detection and diagnosis of prostate disease. Compared to a negative diagnosis (e.g., normal or disease-free), the markers are, variously, more frequently detected, less frequently detected, or differentially detected. The measurement of these markers, alone or in combination, in patient samples provides information that the diagnostician can correlate with a probable diagnosis of prostate cancer, including a probable diagnosis of metastatic prostate cancer. In some embodiments, the biomarkers include those listed in Tables 1 and 2. In other embodiments, the biomarkers include MEKK2 or a fragment or variant thereof.

The invention also provides methods that may be used as an aid in the diagnosis of prostate cancer by detecting these novel biomarkers. The detection and measurement of these biomarkers, alone or in combination, in test samples, provides information that may be correlated with a prognosis of an individual's prostate health. The biomarkers may be characterized by molecular weight. The biomarkers may be resolved from other proteins in a sample by, e.g., mass spectrometry. In some embodiments, the method of resolution involves MALDI-IMS.

In some embodiments, the invention provides a method of diagnosing prostate cancer in a subject, comprising the steps of: (a) obtaining one or more test samples from the subject; (b) detecting the differential expression of at least one protein marker in the one or more test samples, wherein the protein marker is selected from: M3373, M3443, M3488, M4027, M4274, M4355, M4430, M4635, M4747, M4972, M8205, and M10111; and (c) correlating the detection of differential expression of at least one protein marker with a diagnosis of prostate cancer, wherein the correlation takes into account the amount of the at least one protein marker in the one or more test samples compared to a control amount of the at least one protein marker.

In some embodiments, the invention provides a method of diagnosing metastatic prostate cancer in a subject, comprising the steps of: (a) obtaining one or more test samples from prostate tumor tissue of said subject; (b) detecting the differential expression of at least one protein marker in the one or more test samples, wherein the protein marker is selected from: M4030, M5364, M9533, M6186, M3230, M3817, M3245, M9767, M8963, M9091, M9021, and M6344; and (c) correlating the detection of differential expression of at least one protein marker with a diagnosis of metastatic prostate cancer, wherein the correlation takes into account the amount of the at least one protein marker in the one or more test samples compared to a control amount of the at least one protein marker.

The test samples may be from seminal plasma, blood, serum, urine, prostatic fluid, seminal fluid, semen, or prostate tissue, and may be obtained at any time from the subject, including at biopsy or post-surgery.

In some embodiments, the test samples are first and second serial prostate tissue sections. In other embodiments, the methods further comprise the step (d) of staining the first serial prostate tissue section and comparing the stained first serial tissue section to the differential expression of at least one protein marker detected in the second serial prostate tissue section.

### BRIEF DESCRIPTION OF THE FIGURES

FIG. 1. shows that direct tissue mass spectrometric analysis of human prostate tissue reveals cell-specific profiles. Frozen prostate tissue was processed for MALDI imaging and coated with SPA matrix followed by spectra acquisition in linear mode. A) Representative histology image of H & E stained prostate tissue showing areas of prostate adenocarcinoma (T) benign prostate glands (B) and benign stroma (S). B) Resulting average spectra acquired from each region indicated showing characteristic profiles for different cell types. The inset is an expanded view of the mass range m/z 3000-5300 showing differences in the profiles for each cell type. C) MALDI-IMS of a single prostate tissue containing tumor and uninvolved regions. a) H & E image of a tissue specimen containing a defined area of PCa glands and benign glands. Magnified (10×) views of each cell type are shown in the insets. b) Resulting 2D ion density map of the tissue showing high expression of a peak at m/z 4355 in the PCa area (inset is a scan of the tissue after matrix deposition). c) representative spectra from a single spot obtained in the PCa (T) region and from the benign adjacent glandular (B) area displaying differential expression of the ion at m/z 4355. D) MALDI 2D ion density maps of a PCa containing tissues and a benign prostate tissue. Pathology defined regions of PCa (circled) are shown. Areas in red from the resulting MALDI-IMS indicate high expression of m/z 4355. Spectra exported from representative regions of each tissue are shown in the m/z range of 4000-4600 and display the m/z 4355 peak profile (top panel).

FIG. 2 shows that normalized intensity values for m/z 4355 can discriminate tumor from benign tissue. A) Normalized average intensity values for m/z 4355 in different prostate tissue areas. A total of 23 PCa containing tissues, 14 benign adjacent and 31 distal benign tissues were analyzed via MALDI-IMS. The resulting normalized average intensity values for the m/z 4355 peak were plotted for PCa, benign adjacent, and benign distal regions. The boundary of the box closest to zero indicates the 25th percentile, a line within the box marks the median, and the boundary of the box farthest from zero indicates the 75th percentile. Whiskers above and below the box indicate the 90th and 10th percentiles. *p=3.2 E-5 **p=4.0 E-10. B) The predictive power of the putative biomarker to detect PCa tissue areas was tested using the area under the receiver operator characteristic (ROC) curve. Averaged normalized intensity values obtained from MALDI-IMS spectra for PCa tissue were plotted according to Gleason grade C) or pathological stage D). The high grade samples (n=8: from tissues with grade 4+4 (n=4), 4+5 (n=4)) with pathological stage pT3b (n=5) or pT4 (n=3) are included in the plots but were not part of the validation set.

FIG. 3 shows sequence identification of the peptide at m/z 4355. A) Spectra showing the peak profile of m/z 4355 collected by direct acquisition from tissue or from tissue lysates as indicated in each panel in linear or reflectron mode. B) Deconvoluted MS/MS spectra of monoisotopic peak (m/z 4350.4) collected using TOF/TOF LIFT. Peaks attributed to internal fragments are labeled and indicated with arrows.

FIG. 4 shows that on-tissue trypsin digestion of PCa region detects predicted peptides of MEKK2 fragment. A) H & E stained image of PCa tissue with a region of adenocarcinoma circled (top), and MALDI-IMS showing spatial distribution of parent ion at m/z 4355 (bottom). B) After trypsin treatment on the tissue, 4 predicted fragments corresponding to the MEKK2 fragment sequenced (SEQ ID NO:1): (SLQETRKAKSSSPKKQNDVRVKFEHRGEKRILQFPR fragments detected are in bold) were detected in the PCa tissue. Control is an identical section of PCa tissue without trypsin treatment. A benign section of prostate tissue was also trypsin treated. C) Representative spectra from trypsinized (top) and non-trypsinized (bottom) PCa tissue showing the presence of an ion at m/z 631.3 in the trypsin treated tissue only. D) Western blot analysis for expression of MEKK2 in the indicated prostate cancer cell lines and tissue lysates. Western blot of PCa cell lines (30 μg) and tissue lysates (100 μg) was performed using an antibody to the N-terminal region of MEKK2. Actin was used as a loading control.

FIG. 5 shows MEKK2 expression in prostate tissue. A) Immunohistochemical analysis of frozen prostate tissue sections. H&E stained prostate tissue with a region of PCA circled (left panel). MEKK2 stained serial section showing staining in PCa regions and control section without primary antibody (middle panels). Resulting MALDI-IMS 2D ion density map for m/z 4355 indicating high expression in the region of PCa which stained for MEKK2. B) Representative areas of MEKK2 staining from 3 benign (magnification 100×) and 3 PCa tissues (magnification 200×) showing strong expression of MEKK2 in adenocarcinoma cells and little expression in benign glands. C) MEKK2 immunostaining corresponding to m/z 4355 MALDI-IMS expression in two tissue samples. H & E stained serial sections (left panels) are shown for benign (top) and PCa tissue (Gleason 3+3: bottom) with the corresponding MALDI-IMS 2D ion density maps indicating m/z 4355 expression (middle panels). MEKK2 immunostaining is shown in the right panels (magnification 50×).

FIG. 6 shows a comparison of MALDI-IMS to LCM MALDI-TOF. Top panel is a representative spectra generated from 100 prostate adenocarcinoma cells microdisected and extracts analyzed by MALDI-TOF. Bottom panel is a representative spectra generated from MALDI-IMS of the same tissue in an adjacent slice.

FIG. 7 shows that MALDI-IMS utilizing specific m/z values can identify PCa specific regions with prostate. Direct profiling of human prostate tissues revealed specific peaks both over and under-expressed in PCa regions. A) Average spectra showing characteristic profile for PCa regions and benign adjacent regions indicating the over-expression of 2 peaks at m/z 4355 and m/z 4027 and under-expression of m/z 4274. B) Left—H & E stained histological image of a representative tissue containing an area of PCa circled by a pathologist with a 10× view of the cancerous glands (inset). Right—Corresponding images indicating the expression pattern for each peak shown in A), and the combined image for all three peaks.

FIG. 8 shows MALDI 2D ion density maps of PCa versus benign tissue. Tissues obtained from 8 patients undergoing radical prostatectomy were processed for MALDI-IMS. The 2D ion density maps (middle panels) were generated using the expression of m/z 4355 (upper panels) specific to ROIs designated by examination of serial H&E sections (lower panels). The expression level relates to color as indicated in the scale inset. Shown are 4 sections with tumor (a, b, c, d) and 4 sections of benign tissues (e, f, g, h).

FIG. 9 shows sequence identification of m/z 4355 as a fragment of MEKK2. A Mascot search identified the sequence as matching to the PB-1 domain of MEKK2 representing sequence coverage of 36% for this region.

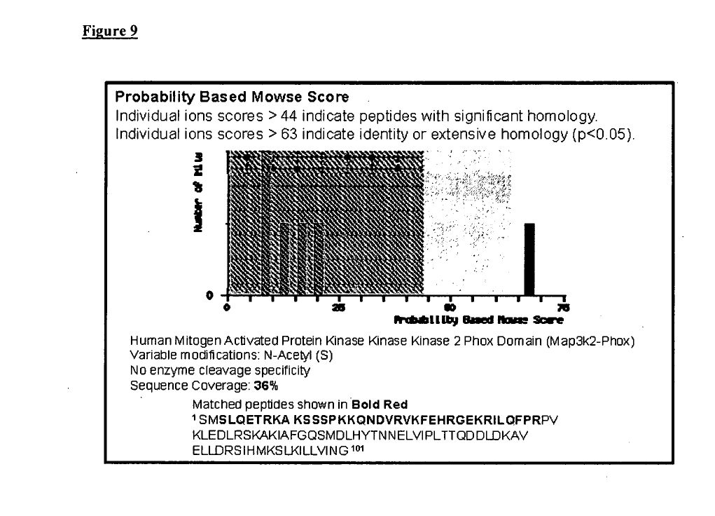

FIG. 10 shows MALDI-IMS analysis of micrometastatic PCa using tissue from involved regions of patients with same risk disease. The “Met” group (1, 2, 3) was subsequently found to harbor metastatic disease, the “Match” group (4, 5, 6) did not. A) shows a MALDI-IMS image using intensity of m/z 4030 (upper panels) and m/z 5364 (lower panels). B) shows the mirror H&E image of Met (3b) and Match (4b) with ROI drawn for involved tissue of the same grade GS 3+4.

FIG. 11 shows MALDI-IMS imaging of UMFix processed prostate tissue. Top panel shows high expression of a peak at m/z 4376 in the area of PCa.

### DETAILED DESCRIPTION OF THE INVENTION

For the purposes of promoting an understanding of the principles of the invention, reference will now be made to certain embodiments and specific language will be used to describe the same. It will nevertheless be understood that no limitation of the scope of the invention is thereby intended, such alteration and further modifications of the invention, and such further applications of the principles of the invention as illustrated herein, being contemplated as would normally occur to one skilled in the art to which the invention relates.

The present invention provides unique markers that are shown herein to be useful in diagnosing or identifying a subject with prostate cancer. The markers of the present invention are shown to be differentially expressed, i.e. either absent/downregulated or upregulated, in a test sample from subjects with prostate cancer as compared to expression in a control sample from subjects known not have prostate cancer. The markers identified herein are shown to distinguish a condition of prostate cancer from benign states, such as normal or non-diseased. The markers identified herein are also shown to distinguish metastatic prostate cancer from non-metastatic prostate cancer. Diagnosis of the metastatic state as disclosed herein may include but is not limited to examination for the presence of specific markers in a test sample from subjects suspected of having a prostate disease. The ability to distinguish different stages of prostate disease has important implications for treatment or management of the subject's condition.

Changes in a prostate tumor or its surrounding tissue may have diagnostic and prognostic utility for disease state. The diagnostic utility of observing changes in excised tissue extends to decisions that are made either at biopsy or post-surgery. Major decisions made at these times are focused upon determining presence and severity of disease at biopsy, and reassessment of disease state after prostate removal.

In certain embodiments, the invention provides biomarkers that aid in the diagnosis of prostate cancer in a subject. Such biomarkers are present in prostate cancer and show high discrimination between cancer versus benign states. The discovery of biomarkers that are differentially present in test samples of prostate cancer and benign states provides important molecular information, for example to the pathologist who is reviewing an image derived from prostate tissue at the time of biopsy or post-surgery and is tasked with providing a diagnosis and staging/grading of disease. By mapping the location of one or more biomarkers that is over-expressed or under-expressed to a tissue region, and comparing the molecular map to a stained mirror tissue, the pathologist's diagnosis of the disease improves in at least two ways: more aggressive cancers and/or associated micrometastatic disease may be identified when the primary tumor appears similar; and a non-specialized pathologist may perform comparably to a specialized pathologist by providing molecular detail to decision making via color-coded mirror tissue.

In certain embodiments, the invention provides biomarkers that differentiate between metastatic and non-metastatic prostate cancer. Such biomarkers may be found in the primary tumor tissue and aid in the discrimination between individuals with metastatic disease and those without metastatic disease. Such biomarkers may also identify “micrometastatic” (hidden) disease in cancer patients, thus rescuing them from radical prostatectomy and redirecting them to appropriate systemic therapy.

### Biomarkers as Prostate Cancer Diagnostics

A biomarker is an organic biomolecule, the presence of which in a sample is used to determine the phenotypic status of the subject or is predictive of a physiological outcome (e.g., prostate health or disease state). In some embodiments, a biomarker is differentially present in a biological sample or fluid taken non-invasively, such as tissue or serum. A biomarker is differentially present between different phenotypic statuses if the mean or median expression level of the biomarker in the different groups is calculated to be statistically significant. Common tests for statistical significance include, among others, t-test, ANOVA, Kruskal-Wallis, Wilcoxon, Mann-Whitney and odds ratio. A single biomarker, or a combination of particular biomarkers, provides measures of relative risk or probability that a subject belongs to one phenotypic status or another. Therefore, they are useful as biomarkers for disease (diagnostics), therapeutic effectiveness of a drug (theranostics), drug toxicity, and predicting and identifying the immune response.

In accordance with the invention, at least one biomarker may be detected. It is to be understood, and is described herein, that one or more biomarkers may be detected and subsequently analyzed, including several or all of the biomarkers identified. Further, it is to be understood that the failure to detect one or more of the biomarkers of the invention, or the detection thereof at levels or quantities that may correlate with a specific state of prostate health, may be useful and desirable as a means of selecting the most favorable treatment regimen, and that the same forms a contemplated aspect of the invention.

The invention provides biomarkers that may be used to distinguish individuals with different states of prostate disease. The biomarkers may be characterized by mass-to-charge ratio as determined by mass spectrometry, by the shape of their spectral peak in time-of-flight mass spectrometry and by their binding characteristics to adsorbent surfaces. These characteristics provide one method to determine whether a particular detected biomolecule is a biomarker of this invention. These characteristics represent inherent characteristics of the biomarkers and not process limitations in the manner in which the biomarkers are discriminated.

The biomarkers of this invention may be characterized by their mass-to-charge (m/z) ratio as determined by mass spectrometry. The mass-to-charge ratio of each biomarker is provided as “M.” Thus, for example, M2454.00 has a measured mass-to-charge ratio of 2454.00. The mass-to-charge ratios are determined from mass spectra generated on any appropriate commercially available mass spectrometer. In some embodiments, the instrument will have a mass accuracy of about +/−0.3 percent. Additionally, the instrument will have a mass resolution of about 400 to 1000 m/dm, where m is mass and dm is the mass spectral peak width at 0.5 peak height. The mass-to-charge ratio of the biomarkers may be determined using appropriate commercially available software. The software assigns a mass-to-charge ratio to a biomarker by clustering the mass-to-charge ratios of the same peaks from all the spectra analyzed, as determined by the mass spectrometer, taking the maximum and minimum mass-to-charge-ratio in the cluster, and dividing by two.

Biomarkers according to the invention include proteins, protein fragments, and peptides. The biomarkers may be isolated from a test sample, such as seminal plasma, blood, serum, urine, prostatic fluid, seminal fluid, semen, or prostate tissue. The biomarkers may be isolated by any method known in the art, based on both their mass and their binding characteristics. For example, a test sample comprising the biomarkers may be subject to chromatographic fractionation, as described herein, and subject to further separation by, e.g., acrylamide gel electrophoresis. Knowledge of the identity of the biomarker also allows their isolation by immunoaffinity chromatography. As used herein, the term “detecting” includes determining the presence, the absence, the quantity, or a combination thereof, of the biomarkers. The quantity of the biomarkers may be represented by the peak intensity as identified by mass spectrometry, for example, or concentration of the biomarkers.

### Detection of Biomarkers

In some embodiments, the invention provides methods for detecting biomarkers. Any one or combination of markers described are within the scope of this aspect of this invention and can be detected. The methods for detecting these markers have many applications. For example, one marker or combination of markers may be detected to aid in differentiating between prostate cancer and a benign state, and thus are useful as an aid in the diagnosis of prostate cancer in a patient. In another example, one marker or combination of markers may be detected to aid in the differentiation between metastatic and non-metastatic prostate cancer. In some embodiments, the marker or markers are detected by gas phase ion spectrometry. In some embodiments, the marker or markers are detected by mass spectrometry and, in particular, laser desorption mass spectrometry.

An energy absorbing molecule (e.g., in solution) can be applied to biomarkers on a probe or substrate. Spraying, pipetting, or dipping can be used. An energy absorbing molecule refers to a molecule that absorbs energy from an energy source in a gas phase ion spectrometer, thereby assisting desorption of markers or other substances from a probe surface. Exemplary energy absorbing molecules include cinnamic acid derivatives, sinapinic acid and dihydroxybenzoic acid.

In some embodiments, a mass spectrometer can be used to detect biomarkers. In a typical mass spectrometer, a probe or substrate is introduced into an inlet system of the mass spectrometer. The analyte is then desorbed by a desorption source such as a laser, fast atom bombardment, or high energy plasma. The generated desorbed, volatilized species consist of preformed ions or neutrals which are ionized as a direct consequence of the desorption event. Generated ions are collected by an ion optic assembly, and then a mass analyzer disperses and analyzes the passing ions. The ions exiting the mass analyzer are detected by a detector. The detector then translates information of the detected ions into mass-to-charge ratios. Detection of the presence of a marker or other substances will typically involve detection of signal intensity. This, in turn, can reflect the quantity and character of a marker on the sample.

In some embodiments, a laser desorption time-of-flight mass spectrometer is used to detect the biomarkers of the invention. In laser desorption mass spectrometry, a probe with a bound analyte is introduced into an inlet system. The analyte is desorbed and ionized into the gas phase by laser from the ionization source. The ions generated are collected by an ion optic assembly, and then in a time-of-flight mass analyzer, ions are accelerated through a short high voltage field and let drift into a high vacuum chamber. At the far end of the high vacuum chamber, the accelerated ions strike a sensitive detector surface at a different time. Since the time-of-flight is a function of the mass of the ions, the elapsed time between ion formation and ion detector impact can be used to identify the presence or absence of molecules of specific mass to charge ratio. As any person skilled in the art will appreciate, any of these components of the laser desorption time-of-flight mass spectrometer can be combined with other components described herein in the assembly of mass spectrometer that employs various means of desorption, acceleration, detection, measurement of time, etc. In another embodiment, an ion mobility spectrometer can be used to detect and characterize a marker. The principle of ion mobility spectrometry is based on different mobility of ions. Specifically, ions of a sample produced by ionization move at different rates, due to their difference in, e.g., mass, charge, or shape, through a tube under the influence of an electric field. The ions (typically in the form of a current) are registered at the detector which can then be used to identify a marker or other substances in the sample. One advantage of ion mobility spectrometry is that it can operate at atmospheric pressure.

Data generated by desorption and detection of markers may be analyzed with the use of a programmable digital computer. The computer program generally contains a readable medium that stores codes. Certain code can be devoted to memory that includes the location of each feature on a probe, the identity of the adsorbent at that feature and the elution conditions used to wash the adsorbent. Using this information, the program can then identify the set of features on the probe defining certain selectivity characteristics (e.g., types of adsorbent and eluants used). The computer also contains code that receives as input, data on the strength of the signal at various molecular masses received from a particular addressable location on the probe. These data can indicate the number of markers detected, optionally including the strength of the signal and the determined molecular mass for each marker detected.

Data analysis can include the steps of determining signal strength (e.g., height of peaks) of a marker detected and removing “outerliers” (data deviating from a predetermined statistical distribution). For example, the observed peaks can be normalized, a process whereby the height of each peak relative to some reference is calculated. For example, a reference can be background noise generated by instrument and chemicals (e.g., energy absorbing molecule) which is set as zero in the scale. Then the signal strength detected for each marker or other substances can be displayed in the form of relative intensities in the scale desired (e.g., 100). Alternatively, a standard may be admitted with the sample so that a peak from the standard can be used as a reference to calculate relative intensities of the signals observed for each marker or other markers detected.

The computer can transform the resulting data into various formats for displaying. In one format, referred to as “spectrum view or retentate map,” a standard spectral view can be displayed, wherein the view depicts the quantity of marker reaching the detector at each particular molecular weight. In another format, referred to as “peak map,” only the peak height and mass information are retained from the spectrum view, yielding a cleaner image and enabling markers with nearly identical molecular weights to be more easily seen. In yet another format, referred to as “gel view,” each mass from the peak view can be converted into a grayscale image based on the height of each peak, resulting in an appearance similar to bands on electrophoretic gels. In yet another format, referred to as “3-D overlays,” several spectra can be overlaid to study subtle changes in relative peak heights. In yet another format, referred to as “difference map view,” two or more spectra can be compared, conveniently highlighting unique markers and markers which are up- or down-regulated between samples. Marker profiles (spectra) from any two samples may be compared visually.

### MALDI-IMS Identification of Biomarkers

In certain embodiments, the invention provides biomarkers that are differentially present in samples of prostate cancer and a benign state identified by the use of MALDI-IMS, or Matrix-Assisted Laser Desorption/Ionization Mass Spectrometric Tissue Imaging. MALDI-IMS is described in, for example U.S. Pat. No. 5,808,300, which is incorporated by reference herein in its entirety.

MALDI-IMS is a technique that allows for imaging of biological samples and has been shown to be quite versatile in its many applications to the analysis of biological samples, such as peptides and proteins. Typically, samples are mixed with an organic compound which acts as a matrix to facilitate ablation and ionization of compounds in the sample. The presence of this matrix is necessary to provide the required sensitivity and specificity to use laser desorption techniques in the analysis of biological material. The application of thin layers of matrix has special advantages, particularly when very high sensitivity is needed.

MALDI-IMS may be used to generate images of samples in one or more m/z pictures, providing the capability for mapping the concentrations of specific molecules in X, Y coordinates of the original biological sample. A MALDI-IMS “image” is achieved by desorption and measurement of tissue proteins/peptides from focused regions, which is subsequently summed across the entire tissue field. Each “spot” is a piece of the composite picture resulting from the grid arrangement of the spots. In this way, a protein/peptide that is overexpressed or underexpressed can have the related expression associated with the tissue region. In effect a region of the tissue that selectively expresses a discrete peptide will display an area of high expression that can be seen from the MS data. In certain embodiments, such images may be matched to a mirror tissue that is reviewed by the pathologist and provides supplemental information to the pathologist to aid in diagnosis and staging/grading of disease.

A set of biomarkers that aids in the differentiation between prostate cancer and non-prostate cancer was identified using MALDI-IMS. These biomarkers are listed in Table 1.

TABLE 1

normalized intensity

| m/z | PCa | Benign | p-value |
|---|---|---|---|
| 3373 | 137.94 | 47.92 | 2.81E−08 |
| 3443 | 297.13 | 139.79 | 7.59E−09 |
| 3488 | 202.59 | 95.09 | 4.38E−08 |
| 4027 | 169.42 | 124.76 | 1.27E−07 |
| 4274 | 104.24 | 147.76 | 7.94E−21 |
| 4355 | 272.3 | 165.51 | 2.76E−16 |
| 4430 | 59.36 | 80.01 | 9.48E−20 |
| 4635 | 88.46 | 131.09 | 7.31E−25 |
| 4747 | 37.43 | 53.77 | 7.31E−25 |
| 4972 | 85.27 | 124.72 | 4.00E−17 |
| 8205 | 35.71 | 62.88 | 1.92E−07 |
| 10111 | 117.34 | 308.82 | 3.34E−05 |

The biomarker of molecular weight 4355 m/z was identified as a peptide fragment of MEKK2, a member of the MAP kinase family. As shown in Example 1, this biomarker correctly differentiated between prostate cancer and benign states.

### MEKK2 and MAP Kinases

In some embodiments, the invention provides a method of diagnosing prostate cancer in a subject, comprising detecting the differential expression of at least one protein marker in the one or more test samples obtained from the subject, wherein the protein marker is MEKK2, a fragment of MEKK2 or a variant of MEKK2.

One of the principal mechanisms by which cellular regulation is effected is through the transduction of extracellular signals across the membrane that in turn modulate biochemical pathways within the cell. Protein phosphorylation represents one course by which intracellular signals are propagated from molecule to molecule resulting finally in a cellular response. These signal transduction cascades are highly regulated and often overlapping as evidenced by the existence of many protein kinases as well as protein phosphatases. It is believed that a number of disease states and/or disorders are a result of either aberrant expression or functional mutations in the molecular components of kinase cascades. Consequently, considerable attention has been devoted to the characterization of these proteins.

Nearly all cell surface receptors use one or more of the mitogen-activated protein kinase (MAP kinase) cascades during signal transduction. Three distinct subgroups of the MAP kinases have been identified and each of these consists of a specific module of downstream kinases. One subgroup of the MAP kinases is the Jun N-terminal kinase/Stress activated protein kinase (JNK/SAPK) cascade. This pathway was originally identified as an oncogene- and ultraviolet light stimulated kinase pathway but is now known to be activated by growth factors, cytokines and T-cell costimulation (19).

MEKK2 (also known as mitogen-activated protein kinase kinase kinase 2, MEK kinase 2 and MAP/ERK kinase kinase 2) is a dual specific serine/threonine kinase that functions to mediate cellular responses to mitogenic stimuli. The MEKK2 protein has, been shown to regulate signaling events associated with two of the three branches of MAP kinase pathways. Originally isolated and cloned from mouse NIH3T3 cells, the human sequence of MEKK2 has also been cloned and identified (20).

MEKK2 has been implicated in inflammatory responses. Zhao et al. have shown that MEKK2 can activate the NF-kappa-B pathway in HeLa cells. NF-kappa-B is a transcription factor that translocates to the nucleus affecting the transcription of several genes upon cellular induction by proinflammatory agents. MEKK2 was shown to induce NF-kappa-B activity by phosphorylating an inhibitor molecule, IkB, that sequesters NF-kappa-B in the cytoplasm. This phosphorylation releases NF-kappa-B for translocation into the nucleus (21). The pharmacological modulation of MEKK2 activity and/or expression may therefore be an appropriate point of therapeutic intervention in pathological conditions.

Biochemical and genetic studies have demonstrated that MAP3Ks are crucial in relaying distinct cell-surface signals through various downstream MAPK pathways. MEKK2 is one of only two of the 20 known MAP3Ks, the other being MEKK3, which regulate the mitogen/extracellular-signal-regulated kinase kinase 5/extracellular signal-regulated kinase-5 (MEK5/ERK5) pathway (22-24). Growth factors and oxidative/osmotic stress have been shown to stimulate the three-tier ERK-5 kinase module consisting of MEKK2/3, MEK5 and ERK5. MEKK2 and MEKK3 encode PB1 domains that have been shown to selectively heterodimerize with the MEK5 PB1 domain to form a functional MEKK2 (or MEKK3)-MEK5-ERK5 ternary complex (22, 24). The ERK5 pathway mediates normal cell-cell interactions during immune surveillance and is a critical regulator of cell invasion during tumor metastasis (reviewed in 25). Indeed, the ERK5 pathway has been implicated in high grade prostate cancer. Specifically, an increase in MEK5 expression was associated with metastatic prostate cancer, cell proliferation, MMP-9 expression and cell invasion (26). Strong MEK5 expression was also found to correlate with the presence of bony metastases and less favourable disease-specific survival. An additional report found significant correlation between ERK5 cytoplasmic overexpression, Gleason sum score and less favorable disease-specific survival (27). It has also been found that ERK5 nuclear expression is significantly associated with the transition from hormone-sensitive to hormone insensitive disease. The finding that MEKK2 is overexpressed in tumor compared to benign is consistent with established biological behavior of ERK5 signaling.

One study that examined the interactions of the MEK5 PB1 domain found that both MEKK2 and ERK5 interact with the N-terminal extension of MEK5, suggesting that MEKK2 and ERK5 compete for binding to MEK5 rather than form a ternary complex (28). The PB1s are dimerization/oligomerization domains which are present in adaptor and scaffold proteins as well as kinases. PB1 domain-dependent MEKK2/3-MEK5 heterodimers provide a spatially organized signaling complex primed to activate ERK5 in response to activation of MEKK2 or MEKK3. No other MAPK cascade has been shown to form such a complex. Interestingly, the m/z 4355 represents a peptide fragment that lies within the PB 1 domain and may reflect molecular pathway changes indicative of PCa development.

Biomarkers of the invention include amino acid sequence variants of MEKK2. These variants may, for instance, be minor sequence variants of the polypeptide which arise due to natural variation within the population or they may be homologues found in other species. They also may be sequences which do not occur naturally but which are sufficiently similar that they function similarly and/or elicit an immune response that cross-reacts with natural forms of the polypeptide. Sequence variants may be prepared by standard methods of site-directed mutagenesis that are well-known in the art.

Amino acid sequence variants of the polypeptide may be substitutional, insertional or deletion variants. Deletion variants lack one or more residues of the native protein which are not essential for function or immunogenic activity, such as variants lacking a transmembrane sequence. Another common type of deletion variant is one lacking secretory signal sequences or signal sequences directing a protein to bind to a particular part of a cell. An example of the latter sequence is the SH2 domain, which induces protein binding to phosphotyrosine residues.

Substitutional variants typically contain an alternative amino acid at one or more sites within the protein, and may be designed to modulate one or more properties of the polypeptide such as stability against proteolytic cleavage. Substitutions may be are conservative, that is, one amino acid is replaced with one of similar size and charge. Conservative substitutions are well known in the art and include, for example, the changes of: alanine to serine; arginine to lysine; asparagine to glutamine or histidine; aspartate to glutamate; cysteine to serine; glutamine to asparagine; glutamate to aspartate; glycine to proline; histidine to asparagine or glutamine; isoleucine to leucine or valine; leucine to valine or isoleucine; lysine to arginine, glutamine, or glutamate; methionine to leucine or isoleucine; phenylalanine to tyrosine, leucine or methionine; serine to threonine; threonine to serine; tryptophan to tyrosine; tyrosine to tryptophan or phenylalanine; and valine to isoleucine or leucine.

Insertional variants include fusion proteins such as those used to allow rapid purification of the polypeptide and also may include hybrid proteins containing sequences from other proteins and polypeptides which are homologues of the polypeptide. For example, an insertional variant may include portions of the amino acid sequence of the polypeptide from one species, together with portions of the homologous polypeptide from another species. Other insertional variants may include those in which additional amino acids are introduced within the coding sequence of the polypeptide. These typically are smaller insertions than the fusion proteins described above and are introduced, for example, to disrupt a protease cleavage site.

A set of biomarkers that aids in the differentiation between metastatic and non-metastatic prostate cancer tissue was also identified using MALDI-IMS. These biomarkers are listed in Table 2 and described in Example 2.

TABLE 2

Normalized intensity

| m/z | Mets | No-Mets | p-value |
|---|---|---|---|
| 4030 | 61.41 | 41.18 | 2.59E−88 |
| 5364 | 61.72 | 42.96 | 3.97E−83 |
| 9533 | 21.32 | 8.4 | 1.13E−79 |
| 6186 | 83.43 | 45.48 | 8.75E−64 |
| 3230 | 31.93 | 65.12 | 6.79E−56 |
| 3817 | 35.17 | 59.12 | 1.57E−52 |
| 3245 | 24.86 | 36.88 | 1.17E−51 |
| 9767 | 25.07 | 8.85 | 3.56E−47 |
| 8963 | 42.24 | 10.78 | 3.59E−46 |
| 9091 | 50.71 | 14.39 | 8.89E−45 |
| 9021 | 17.39 | 6.28 | 1.39E−42 |
| 6344 | 37.64 | 31.13 | 5.49E−39 |
| [symbol] | | | |

[symbol]

indicates data missing or illegible when filed

The markers of the invention identified by MALDI-IMS can be detected by other methods, also within the scope of the invention. Such methods may include chromatographic methods, such as liquid chromatography or gel chromatography, or immunoassays.

Using the purified markers or their nucleic acid sequences, antibodies that specifically bind to a marker can be prepared using any suitable methods known in the art. See, e.g., Current Protocols in Immunology (2007); Harlow & Lane, Antibodies: A Laboratory Manual (1988); Goding, Monoclonal Antibodies: Principles and Practice (3d ed. 1996); and Kohler & Milstein, Nature 256:495-497 (1975). Such techniques include, but are not limited to, antibody preparation by selection of antibodies from libraries of recombinant antibodies in phage or similar vectors, as well as preparation of polyclonal and monoclonal antibodies by immunizing rabbits or mice (see, e.g., Huse et al., Science 246: t275-1281 (1989); Ward et al., Nature 341:544-546 (1989)).

After the antibody is provided, a marker can be detected and/or quantified using any of a number of well recognized immunological binding assays (see, e.g., U.S. Pat. Nos. 4,366,241; 4,376,110; 4,517,288; and 4,837,168). Useful assays include, for example, an enzyme immune assay (EIA) such as enzyme-linked immunosorbent assay (ELISA), a radioimmune assay (RIA), a Western blot assay, or a slot blot assay. For a review of the general immunoassays, see also, Methods in Cell Biology: Antibodies in Cell Biology, volume 37 (Asai, ed. 1993); Basic and Clinical Immunology (Stites & Teff, eds., 7th ed. 1991).

Reference will now be made to specific examples illustrating the constructs and methods above. It is to be understood that the examples are provided to illustrate preferred embodiments and that no limitation to the scope of the invention is intended thereby.

### EXAMPLES

### Example 1

### Identification of Prostate Cancer Tumor-Specific Biomarkers

### A. Materials and Methods

Patients and Tissue Samples. Patients were consented prior to undergoing radical prostatectomy at Sentara Norfolk General Hospital. Study protocols were approved by the institutional review board at EVMS. The age range of the patients was 46-82 with a mean age of 58.8 years. A total of 75 patients (21 for the discovery set and 54 for the validation set) were recruited for this study. Two cored specimens were harvested from each prostate immediately after removal of the gland. Each core is divided longitudinally to create mirrored cores; one was fixed and paraffin embedded and the other is embedded in OCT (optimal cutting temperature compound, Sakura Finetek USA) and frozen at 80° C. The frozen blocks yielded 41 sections (10 for the discovery set and 31 for the validation set) of benign tissue harvested from prostate tissue distal from the tumor site and 34 sections (11 for the discovery set and 23 for the validation set) of PCa containing tissue. Of the 23 PCa containing blocks in the validation set, 14 sections harbored benign tissue adjacent to PCa. These were also included as “benign adjacent” samples in the validation set. Cryo-sectioning was performed on a Microm HM 505E cryostat at −20° C. A serial cryosection at 8 μm was stained with hematoxylin and eosin as a guide, and analyzed by a pathologist to determine tissue morphology. Two additional serial sections at 10 μm were mounted on conductive Indium-Tin Oxide (ITO) coated glass slides (Bruker Daltonic, Billerica, Mass.) and used for MALDI-IMS.

Materials. Acetonitrile, ethanol, high-performance liquid chromatography (HPLC) grade water, 3,5-dimethoxy-4-hydroxycinnamic acid (sinapinic acid-SPA) were purchased from Sigma Chemical Co. (St. Louis, Mo.). The α-cyano-4-hydroxycinnamic acid (HCCA) was purchased from Bruker Daltonic. Tri-fluoroacetic acid (TFA) was purchased from Pierce Biotechnology, (Rockfold, Ill.). Rabbit monoclonal antibody to MEKK2 (EP626Y) was purchased from Abcam, Cambridge, Mass.

Tissue section preparation. Immediately after sectioning, the ITO coated slides were washed and fixed with 70% ethanol and 95% ethanol for 30s each (29). A water wash was performed to remove residual embedding media followed by a repeat of the ethanol washes of 70% and 95%. Slides were air dried and stored in a dessicator for 1 hour before matrix deposition. A matrix solution of sinapinic acid (10 mg/ml) containing 75% acetonitrile and 0.13% TFA was sprayed uniformly over the tissue using an automated spraying device, (ImagePrep workstation Bruker Daltonics) which controls matrix deposition and thickness of the matrix layer. Digital images of the sprayed tissue sections were acquired with a flatbed scanner prior to MALDI analysis.

MALDI-IMS analysis and Image processing. Spectra were collected across the entire tissue area using the Ultraflex III MALDI-TOF/TOF instrument (Bruker Daltonics) with a SmartBeam laser operating at 200 Hz in linear mode over a mass range of 2,000 to 45,000 Daltons. A laser spot diameter of 50 μm and a raster width of 100 μm were employed. Using the FlexImaging software (Bruker Daltonic), teaching points were generated to ensure the correct positioning of the laser for spectral acquisition. The software exports the specific geometry of the tissue to be analyzed and a instrument specific automated method is created which generates a grid across the tissue of spots where the laser will acquire data. A total of 200 laser shots were accumulated and averaged from each laser spot rastered across the tissue section. Calibration was performed externally using a peptide standard in the mass range of 700-4500 Da. The intensity of each signal over the entire mass range acquired is plotted as a function of location on the tissue allowing the visualization of the location of each m/z detected. These images were generated and visualized using Flex Imaging and Biomap software (available as free software from www. MALDI-IMS.org.)

Data processing and statistical analysis. Automated analysis of the spectral data was performed to identify all differentially expressed peaks between cell types. Spectra derived from pathology-defined Regions of Interest (ROI) in each tissue were exported using the FlexImaging software for profile analysis. Base-line subtraction, normalization (to total ion current), peak detection, and spectral alignment were performed using ClinProt. A mass window of 0.3% and a signal to noise ratio of 3 were selected for peak detection. A genetic algorithm (GA) using k-nearest neighbors (KNN) was used to obtain a classification between normal and PCa containing tissue. The result of the GA is the peak combination which is proved to separate best between the different classes. Significant differences between groups were determined by Student's t test. A P-value of less than 0.01 was considered to indicate statistical significance. The predictive power of the putative biomarker to detect PCa tissue areas was tested using the area under the receiver operator characteristic (ROC) curve using SPSS for Windows. The optimal cut-off point was defined as that point on the ROC curve that maximizes both sensitivity and specificity.

Tissue and cell culture lysates. Tissue lysates were prepared from bulk frozen prostate tissue (˜0.5 mm³) by homogenizing the samples in a small dounce tube on ice with a solution of 20 mM Hepes, 1% TritonX 100 (1 ml). Lysates were then sonicated at room temperature for 15 minutes and spun down at 14,000 rpm for 2 minutes to remove cellular debris. The lysates were then subjected to fractionation using weak cationic exchanger (WCX) magnetic beads (Bruker Daltonic) according to the suppliers specifications. The bound peptides and proteins were eluted in 20 μl. Five microliters of this eluate was lyophilized and resuspended in 5 μl of HCCA matrix in 50% acetoniltrile with 0.1% TFA. Lysates from the prostate cancer cell lines (Du145, LnCap and PC-3) were prepared from 10⁶ cells in a lysis buffer containing 0.3% SDS, 3% DTT and 30 mM Tris pH 7.5.

MALDI-MS/MS and Protein Identification. One microliter of each tissue lysate mixed with matrix was then spotted on a steel MALDI target. The mass profiles were recorded by MALDI MS using the same acquisition parameters as for tissue imaging. Data was collected on the UltraFlex III in reflectron mode to verify the presence of the peak of interest. MS/MS analysis of the peak was then performed in LIFT mode. An optimized high mass LIFT method was used and externally calibrated with fragments from a peptide standard with parent masses in the mass range of 700-4500 Da. A parent mass (monoisotopic mass as determined in reflectron mode) was selected and LIFT analysis (MS/MS) was performed in the Ultraflex TOF-TOF. Peaks were labeled using FlexAnalysis software and opened in BioTools 3.1 (Bruker Daltonic). The MS/MS spectrum sequence analysis and database search was performed using MASCOT 2.2.03 with the following settings: MS Tol.: 70 ppm, MS/MS Tol.: 1.0 Da, no enzyme designation, serine acetylation, using the National Center for Biotechnology Information database for human sequences with 20,080,125 entries.

Trypsin digestion was performed on the tissue slices (10 μm) by spotting 0.5 μl of a solution of 0.769 μg/ul trypsin in 50 mM Ammonium bicarbonate pH. 8.0. The tissue slices were then incubated at 37° C. for 2 hours in a humidity chamber. Following trypsin treatment the tissue sections were spray coated with HCCA (7 mg/ml in 50% acetonitrile, 0.2% TFA). Data was collected across the tissue in reflectron mode and converted to BioMap images.

Immunohistochemistry Immunostaining of frozen specimens was performed by the avidin-biotin peroxidase complex method using a Vectastain Elite ABC kit (Vector, Burlingame, Calif.). Blocking was performed with an avidin-biotin blocking kit (Vector Laboratories, catalog number SP-2001). Fixed and Frozen tissues were then treated with 0.3% hydrogen peroxide to block endogenous peroxide activity for 15 minutes. Sections were incubated in normal goat serum to block nonspecific binding and incubated with rabbit monoclonal antibody to MEKK2 (EP626Y: Abcam, Cambridge, Mass.) diluted 1:50 in PBS for 1 hour at room temperature. This antibody reacts with the N-terminal portion of MEKK2. Sections were then treated with biotinylated goat anti-rabbit immunoglobulin G (IgG), followed by treatment with avidin-biotin-peroxidase complex, and stained with IMPACT DAB peroxidase substrate (Vector Labs) according to the suppliers protocol. Counterstaining was performed with Mayer's hematoxylin.

Western Blot Analysis. A total of 30 μg (for cell lysates) or 100 μg (for tissue lysates) of protein was separated on a 4-12% SDS-PAGE gel and transferred by semi-dry transfer method to PVDF membranes (Immobilon-P, Millipore, Billerica, Mass.). The membranes were then incubated in Odyssey™ blocking buffer diluted 1:1 in PBS for 1 hour at room temperature. Incubation with rabbit monoclonal antibody to MEKK2 (EP626Y) diluted 1:1000 in blocking buffer was performed overnight at 4° C. with gentle shaking. Blots were then washed four times with 5 min incubations in PBS and 0.1% Tween 20 (PBST). The secondary antibody, Alexa Fluor IRDye 800 cw goat anti-rabbit IgG (# 926-32211), was diluted 1:5000 with blocking buffer, 0.1% Tween 20, and 0.01% SDS. The membrane was incubated with 10 ml of this solution with gentle rotational mixing at room temperature for 1 h protected from light. The membranes were then washed in PBST as before, rinsed in PBS and scanned with the Odyssey infrared imaging system (LI-COR, Lincoln, Nebr.).

### B. Results

Identification of an Expression Profile that Discriminates between PCa and Adjacent Normal Tissue. The investigation into the presence of a prostate cancer specific protein/peptide expression profile was conducted on a total of 75 prostate tissue samples. The patient and tissue sample characteristics of the discovery and validation cohort are presented in Table 3. Tissue sections were uniformly coated with matrix using an automated spraying device, and adjacent serial sections were stained with hemotoxylin and eosin for histopathology. Parallel stained slides of each section were read by a genitourinary-trained pathologist and the regions of interest (ROI) designated. These ROI contained prostate adenocarcinoma cell populations, benign adjacent epithelial cells, as well as stromal and benign epithelial cells from tissue specimens without tumor cells present.

TABLE 3

| Characteristic | Discovery | Validation |
|---|---|---|
| PCa tissues | | |
| Patients | 11 | 23 |
| mean age (range) | 59.4 (46-65) | 58.5 (46-82) |
| Mean preoperative PSA | 7.97 (4.0-13.3) | 11.1 (2.4-69.6) |
| ng/ml (range) | | |
| Pathological classification | | |
| PT2a | 0 | 3 |
| PT2b | 0 | 1 |
| PT2c | 7 | 8 |
| PT3a | 4 | 8 |
| PT3b | 0 | 3 |
| Gleason score (section tested) | | |
| 3 + 3 | 9 | 9 |
| 3 + 4 | 2 | 10 |
| 4 + 3 | 0 | 4 |
| Total patients\total sections | 11\11 | 23\23 |
| tested | | |
| Benign tissues* | | |
| Patients | 10 | 31 |
| mean age (range) | 58.4 (51-65) | 58.8 (46-76) |
| Mean preoperative PSA | 8.30 (4.8-15.5) | 10.8 (3.6-51.8) |
| ng/ml (range) | | |
| Pathological classification | | |
| PT2a | 1 | 4 |
| PT2b | 1 | 1 |
| PT2c | 5 | 8 |
| PT3a | 3 | 12 |
| PT3b | 0 | 6 |
| Total patients\total sections tested | 10\10 | 31\31 |
| Total Patients | 21 | 54 |
| Total tissue sections | 21 | 54 |

Peptides Differentially Expressed

| m/z | p value |
|---|---|
| Highly Expressed in PCa cells | |
| 3373 | 2.81E−08 |
| 3443 | 7.59E−09 |
| 3488 | 4.38.E−08 |
| 4027 | 1.27E−07 |
| 4355 | 2.76E−16 |
| Highly Expressed in Benign cells | |
| 4274 | 7.94E−021 |

| Correct classification | Discovery set | Validation set |
|---|---|---|
| PCa areas | 85% | 81% |

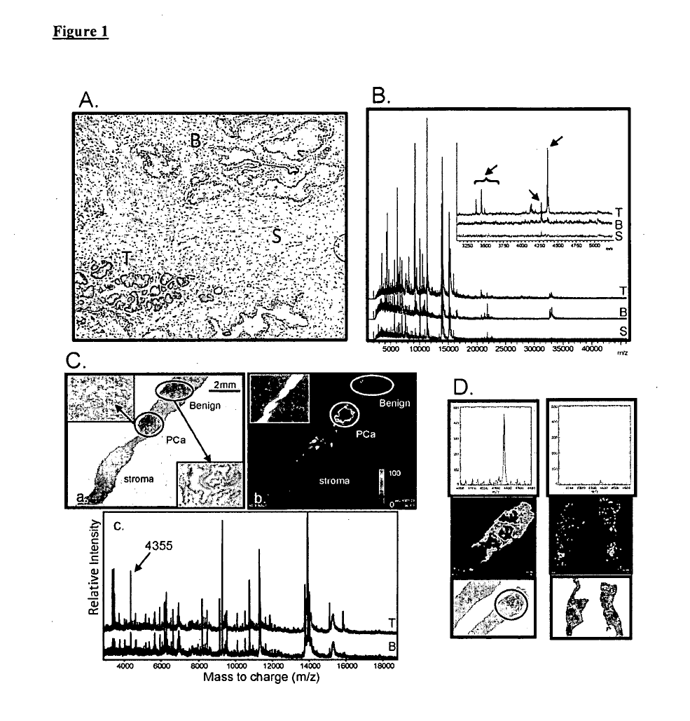

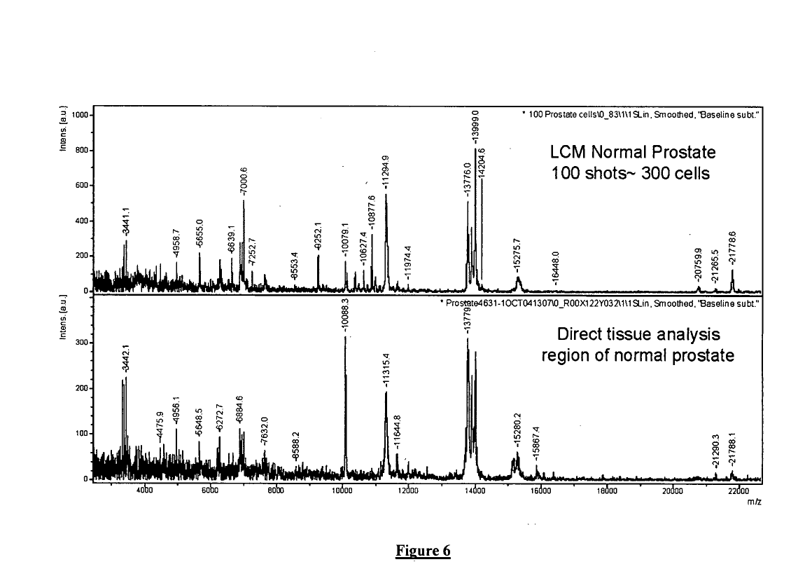

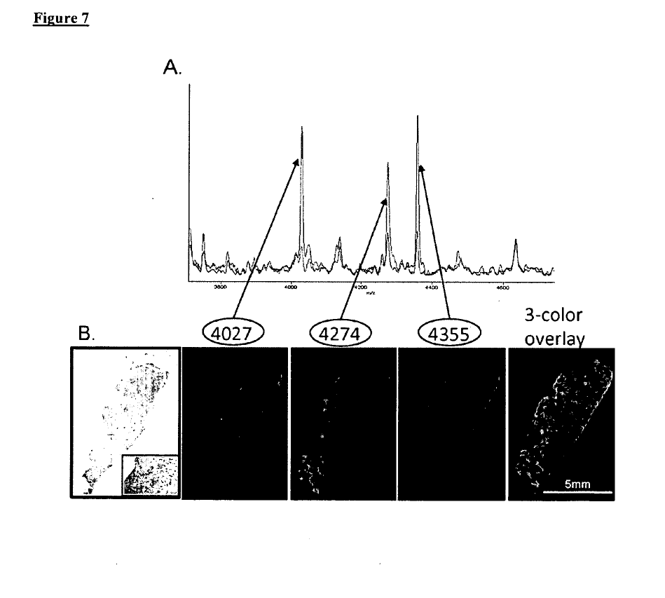

In the initial discovery experiment 21 tissue sections (11 PCa and 10 benign) were analyzed. The resulting spectra were used to generate two-dimensional molecular maps of the peptides and proteins present in each tissue section and automated analysis of the spectral data was performed to identify differentially expressed peaks. The resolving power of the technique was comparable to LCM capture of cells followed by MALDI-TOF analysis of the extracted proteins (FIG. 6). On average between 350 and 400 peaks within the mass range m/z 2,000 to 20,000 could be resolved. Table 1 is a list of the top candidate proteins that were differentially expressed between the tissue types. Several peptide ions were found to discriminate PCa from benign tissue (Table 3). Examination by a pathologist revealed specific regions with designated cell types present in prostate tissue sections (FIG. 1A). This process is defined as pathology designated ROI. In the mass range m/z 3000-5000 several differentially expressed ions were detected which could be used to discriminate between PCa and adjacent benign regions (FIG. 1B inset and Table 3). Two peptide ions at an average m/z of 4027 and 4355 showed significant over-expression in PCa cells when compared to benign adjacent cells spectra. Three ions over expressed in PCa (m/z=3,372, 3,443, and 3,487) are consistent in mass with defensin peptides and may be indicative of infiltrating neutrophils (30, 31). Another peak at m/z 4274 was expressed in benign adjacent epithelial cells and stroma with little or no expression seen in PCa cells. Spectra derived from ROIs designated as tumor or benign from the initial 21 tissues examined were used to generate a classification algorithm using three m/z values (m/z 4027, 4274, 4355) which was capable of correctly classifying 85% of PCa tissue areas. Selected component ions with significant discriminating power were evaluated in these initial tissues and images derived around the pathology-designated ROI. This allowed for a visual determination of region specific changes in peptide ion expression. Representative examples of images derived from mapping all three discriminating m/z are shown in FIG. 7.

The classification ability of the same genetic algorithm derived from the discovery set utilizing the three discriminatory peaks (m/z 4027, 4274, 4355) was then evaluated upon the larger validation set. This set consisted of 23 tumor sections and 31 benign sections for a total of 54 sections. The performance of the three-peak genetic algorithm in the validation set was comparable to that seen in the discovery set. Specifically, the PCa areas in the validation set could be correctly classified in 81% of the tissues tested (Table 3).

MALDI-IMS Utilizing m/z 4355 can Identify Pca Specific Regions of Prostate. From the list of differentially expressed peaks from the initial discovery set of 21 tissues, the ion at m/z 4355 was the most significantly over-expressed, in PCa containing tissue regions (p=2.76×10⁻¹⁶). Further evaluation of the utility of this peak alone for the detection of PCa regions within prostate tissue was performed via MALDI-IMS. FIG. 1C is a representative image of a tissue section with one specific region of PCa cells and clearly defined adjacent regions of normal prostate glands. A higher magnification view of each cell type can be seen in the insets. Clearly evident from the ion density map, the m/z 4355 peak was highly expressed in the PCa region as compared to the surrounding tissue. Little to no expression is visible in the normal stroma or benign glandular regions. When representative spectra were exported from the specific regions (tumor vs. benign) a prominent peak at m/z 4355 was clearly observed to be over-expressed in the PCa obtained profile.

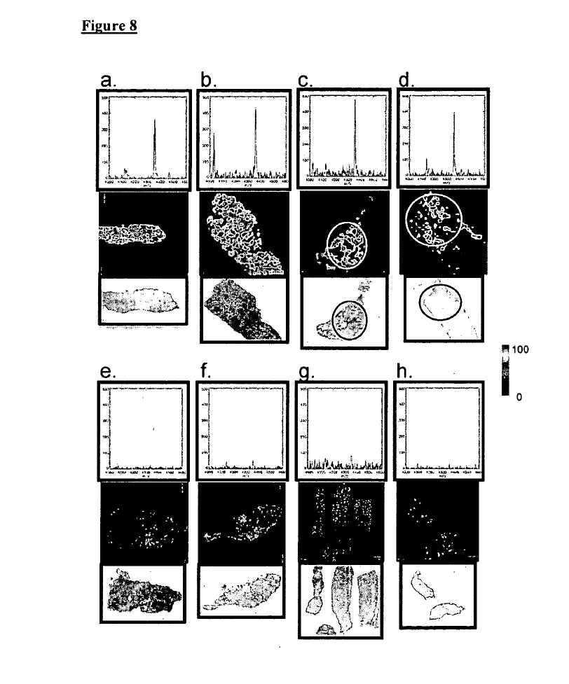

MALDI-IMS Utilizing m/z 4355 Discriminates between Cancer and Uninvolved Prostate Tissue. In order to evaluate the differential expression of m/z 4355 between PCa and benign regions, an analysis of the validation set of prostate tissues was conducted. The images produced from the ion density of the m/z 4355 peak following the analysis of 23 PCa and 31 benign prostate tissue sections (distal from tumor site) were examined, in addition to 14 benign prostate tissue regions found adjacent to tumor in 14 of the 23 PCa sections. Shown in FIG. 1D are representative ion images of the expression of the m/z 4355 peak in tissues containing PCa and distal benign sections. The corresponding spectra in the representative region of m/z 4000-4600 are shown in the top panels above each image. A set value for the peak intensity threshold used to display the m/z 4355 peak in each image was determined from the discovery set and applied to the validation set. This threshold represented the maximum peak intensity observed from the normalized intensity values obtained in PCa regions. Any pixel displaying an intensity greater than or equal to this set threshold was then considered high expression and is represented in the images obtained in the validation set in each image. An intensity scale can be seen at the bottom right of FIG. 1C. Provided in FIG. 8 is a representative set of 8 paired tissues. High expression was visible in each section where PCa cells were present or visible throughout the tissue when no benign cells were present. In contrast little to no expression of the m/z 4355 ion was detected in sections containing benign cells only.

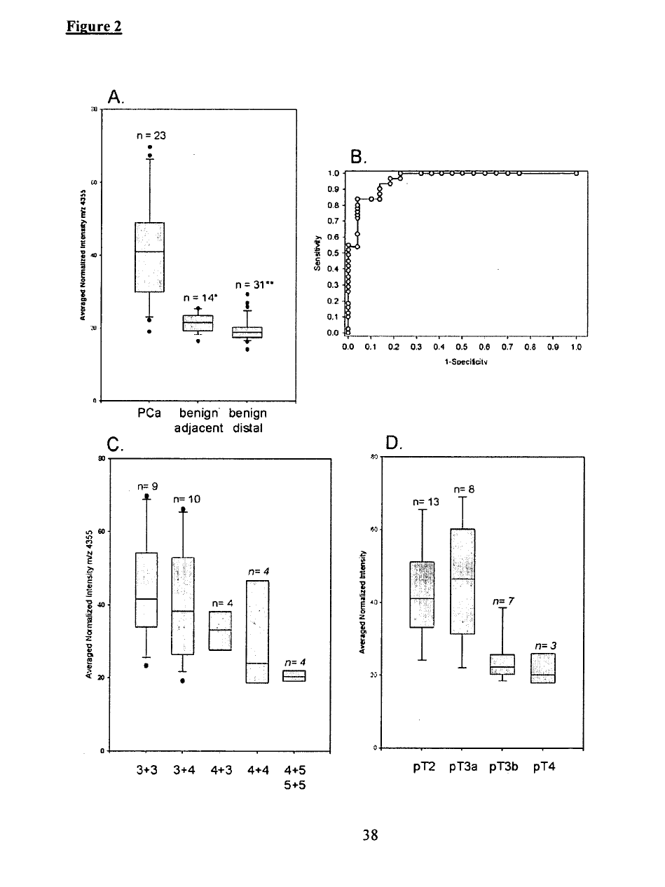

In order to illustrate the tissue specific expression of m/z 4355, the intensity values with respect to defined tissue regions across the separate validation sample set were examined. Intensity values for m/z 4355, normalized to total ion current, were calculated for each tissue region and plotted; PCa, benign adjacent, and benign distal (FIG. 2A). The average normalized intensity of the m/z 4355 peak in benign tissue found in the same section with PCa cells or benign prostate tissue from a section containing no PCa cells was 20.8 and 19.6 respectively, whereas for PCa regions the average intensity found in PCa tissue was 41.1, representing a 2.1 fold increase. A ROC curve calculated from the average normalized intensity of each ROI (distal benign vs PCa) is shown in FIG. 2B. The optimal cutoff point for using the 4355 peak as a biomarker for PCa in tissue sections was a normalized average intensity value of 33. This cutoff point was associated with a sensitivity of 90.3% and a specificity of 86.4% (AUC 0.960). In order to maximize sensitivity a cut-off value of 23.8 was chosen which represents a sensitivity of 96.8% and a specificity of 81.8%.

A more detailed analysis of the expression of the m/z 4355 by disease stage/grade was also conducted. The normalized intensity values for m/z 4355 were plotted by Gleason grade and pathological stage. The results of this analysis can be seen in FIGS. 2C and 2D. Tissues with a Gleason combined score of 3+3 had an averaged normalized intensity value for m/z 4355 of 44.8 with 89% of the tissues exhibiting a value above the ROC cut-off of 23.8. Tissues with a Gleason score of 3+4 had an averaged normalized intensity value of 41.0 with 90% of these tissues displaying a value above the ROC cut-off. Tissues with a Gleason score of 4+3 had an averaged normalized intensity of 32.0 with 75% of tissues displaying a value above the ROC cut-off. This reduction in expression of the m/z 4355 peak observed with increasing Gleason grade was also observed between pathological stage. While this reduction was not significant between Gleason scores, a significant reduction was seen between pathological stages pT2 and pT3b as well as pT3a vs. pT3b. Tissues from prostates designated as pT2a, b or c had a normalized average intensity of 42.7 with 92.3% of the tissue samples above the cut-off. A similar trend is seen in tissues from prostates designated pT3a which had an average intensity of 45.6 for m/z 4355 with 87.5% of the tissues with a value above 23.8. If however, the tissue specimen was procured from a prostate with a pathological designation of pT3b indicating seminal vesicle invasion, the average normalized intensity dropped to 30.1 with only 28.6% of tissues having a value above the cut-off.

In order to further define the trend of decreasing expression of the m/z 4355 with increasing stage/grade of disease, an additional 8 sections from more aggressive disease (Gleason 8/9/10 and pT4) were examined. The analysis was conducted as described above for the previous tissues and the evaluation of m/z 4355 was conducted with the same cut-off score. As can be seen in FIGS. 2C and D, the trend toward decreased expression was observed in higher grade/stage disease. Specifically, of the 8 high grade tissues tested, only 2 (25%) had expression above the cut-off value of 23.8. Of the high grade cases, three were designated as pT4 and only 1 (33%) of these had a normalized intensity value for m/z 4355 above the cut-off.

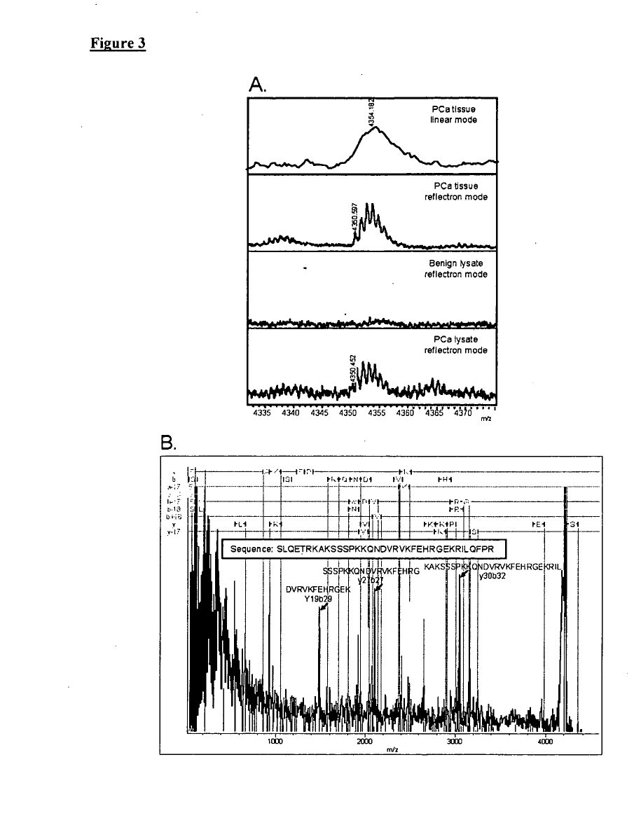

Sequence Identification of m/z 4355 as a fragment of MEKK2. Having established the differential expression of a peptide ion at m/z 4355, the sequence of the peptide was identified. Lysates were prepared from 4 tissue samples: 2 PCA and 2 benign prostate. Sections from these tissues were examined in the initial MALDI-IMS analysis and found to have high or low expression of the peak at m/z 4355. Protein lysates were incubated with weak cationic magnetic beads, and eluted fractions were shown to be enriched for the m/z 4355 peak as measured by MALDI-TOF. As seen in FIG. 3A, the peptide ion captured from the WCX fractionation of the PCa tissue lysate matches the mass detected directly from the tissue within an error of 0.26 Da, whereas no peptide ion was detected at this m/z in the enriched lysate from benign tissue. The lysate was then concentrated via lyophilization and prepared for MS/MS analysis as described. FIG. 3B shows the fragmentation pattern of the parent ion (m/z 4350.4), in which it can be seen that the spectra contained many large internal fragments observable in the TOF/TOF analysis. The fragmentation series gave a MASCOT top score of 67 (1.0 Da, 70 ppm), with scores ≧63 indicating extensive homology, and matched to a fragment of MEKK2 (Swiss-Prot entry Q9Y2U5) with S-acetylation at the N-terminal of the peptide (FIG. 7). Out of 129 total observed peaks, 126 could be accurately assigned to theoretical fragments of MEKK2. This sequence represents amino acid residues 26-61 in the 619 amino acid full-length sequence and lies within the PhoX-and-Bem1 (PB1)-domain of the molecule.

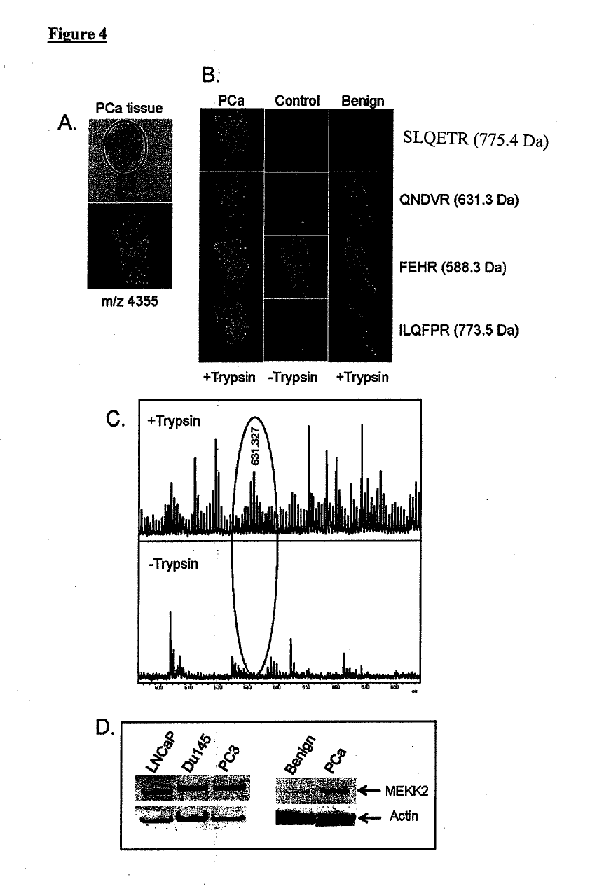

In order to further establish that the m/z 4355 ion derived from tissue is a fragment of MEKK2, an in-tissue digest to analyze for the presence of predicted MEKK2 tryptic fragments was performed. A tissue section previously found to have high expression of the m/z 4355 peak in a PCa region was used for this analysis. Serial sections of the same tissue region were harvested and analyzed. One of the mirror sections was trypsin treated while the adjacent mirror section was untreated and used as a control. Ion density maps were also generated using the indicated theoretical tryptic peptides. As seen in FIG. 4, specific theoretical masses of the predicted MEKK2 fragment after trypsinization could be detected in the trypsin treated PCa tissue. The in-tissue trypsin-generated fragments matching the theoretical digest masses were not present in the mirror untreated section of the same PCa tissue (FIG. 4C). Furthermore, trypsin digestion of benign tissue sections did not generate fragment ions corresponding to theoretical MEKK2 cleavage products (FIG. 4B). A representative ion density map image derived from the parental 4355 m/z is shown as a comparison to images derived from the tryptic peptides (FIG. 4A). This analysis demonstrates that both the parental peptide and predicted peptide fragments display concordant tissue expression.

Expression of MEKK2 in Prostate Cancer Cell Lines and Tissues. Western blot analysis was performed on PCa and benign tissue extracts and 3 prostate cancer cell lines (Du145, LnCap, PC-3). LNCaP cells originate from a lymph node metastatic lesion of human PCa and Du145 and PC-3 are human prostate adenocarcinoma cell lines metastatic to the brain and bone respectively. The antibody used was the same as that employed in the above immunohistochemistry analysis for MEKK2 expression. The relative expression of MEKK2 in these systems is shown in FIG. 4D. All three prostate cell lines showed strong expression of full length MEKK2 (70 kDa). This analysis revealed higher MEKK2 expression in the PCa tissues when compared to the expression seen in the benign tissues. Densitometry analysis indicated a 4.4 fold increased expression of MEKK2 in PCa tissue compared to benign tissue.

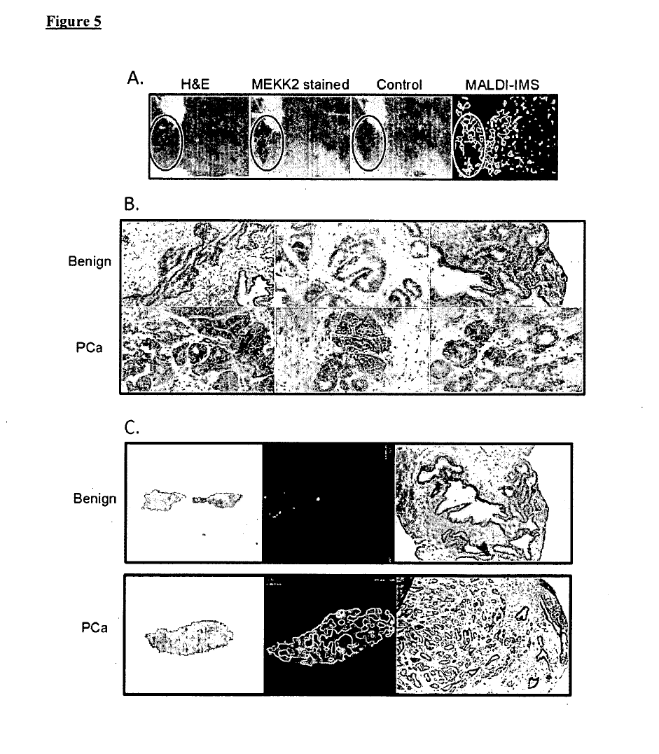

MEKK2 is Overexpressed in PCa Specific Regions of the Prostate. In some cases, the over-expression of a fragment of a protein may coincide with over-expression of the whole protein. The expression of MEKK2 in PCa tissue was examined using immunohistochemistry specific for expression of MEKK2. PCA containing and adjacent uninvolved frozen tissues were stained for MEKK2 expression. The antibody used is specific for the N-terminal portion of the MEKK2 protein where the PB1 domain is located (32). As seen in FIG. 5A, MEKK2 staining correlates with the presence of PCa in the tissue section. The ROI designated in the H&E panel was prescribed by a GU specialized pathologist as containing tumor. Additional prostate tissues were also stained and magnified views of the stained PCa glands and benign tissue can be seen in FIG. 5B. In FIG. 5C, an analysis of sections designated as all tumor or all benign is shown. The prostate tissues examined showed high levels of MEKK2 within involved tissue with predominantly cytoplasmic expression pattern. In contrast, benign glands displayed little to no MEKK2 expression.

### Example 2

### Differential Protein Expression Using MALDI-IMS for the Detection of Metastatic Disease

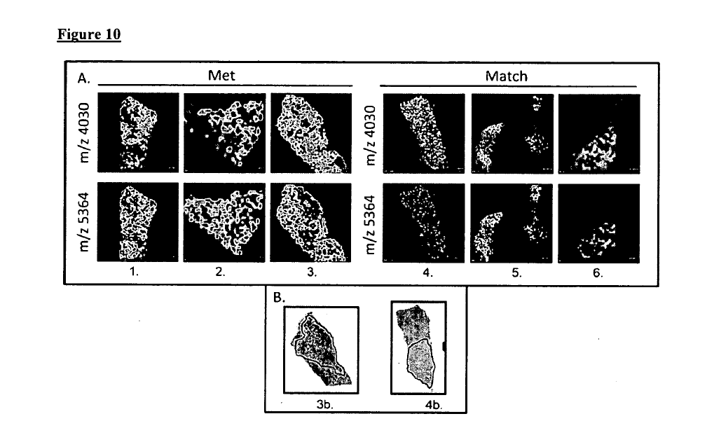

Frozen prostate sections of similar stage disease were processed in which the case/control design is the discovery of metastatic disease after surgery in the case group. This study concentrated on differential expression patterns of the tumor tissue regions between case and control. From the examination of eight pairs of case/control samples, a list of top discriminating spectral peaks was generated and is presented in Table 2. Several of the markers were plotted for image generation and the images were subsequently compared to the mirrored histologically stained and pathology-read sections. FIG. 10 shows the ability to detect cancer in tumors associated with distal metastatic disease using expression of select markers.

### Example 3

### Utilization of UMFix Treated Tissue for MALDI-IMS

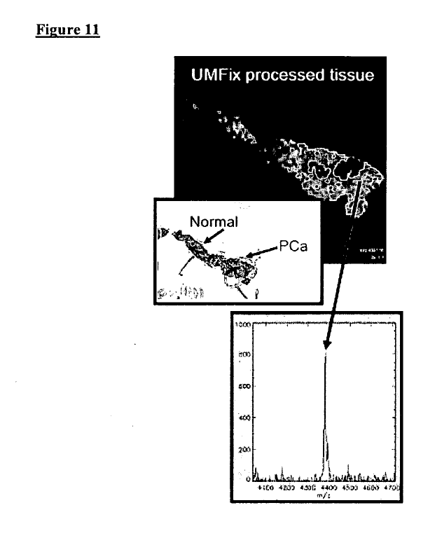

The utility of the inventive biomarkers is evident at the biopsy stage. Since frozen sections are not suitable for biopsy-driven diagnostics, a pathology-friendly fixation method known as UMFix is incorporated that is acceptable to clinical histology but also able to preserve protein. The UMFix approach was developed at the University of Miami and is a commercially viable system for tissue preparation in place and is available to pathologists. The UMFix process preserves both IMS detail and information in a fixed tissue for mirrored histology. FIG. 11 is an image of the profile within the region of the 4360 m/z peak utilizing UMFix preserved tissues.

While the invention has been illustrated and described in the figures and foregoing description, the same is to be considered as illustrative and not restrictive in character, it being understood that only the preferred embodiments have been shown and described and that all changes and modifications that come within the spirit of the invention are desired to be protected. In addition, all references and patents cited herein are indicative of the level of skill in the art and hereby incorporated by reference in their entirety.

### REFERENCES

1. Ries L A G MD, Krapcho M, Stinchcomb D G, Howlader N, Horner M J, Mariotto A, Miller B A, Feuer E J, Altekruse S F, Lewis D R, Clegg L, Eisner M P, Reichman M, Edwards B K (eds). SEER Cancer Statistics Review, 1975-2005. National Cancer Institute Bethesda, Md. 2008; http://seer.cancer.gov/csr/1975_{—}2005/, based on November 2007 SEER data submission, posted to the SEER web site.
2. Nelen V. Epidemiology of prostate cancer. Recent Results in Cancer Research 2007; 175: 1-8.
3. Thompson I M, Pauler D K, Goodman P J, et al. Prevalence of prostate cancer among men with a prostate-specific antigen level <or =4.0 ng per milliliter. [see comment] [erratum appears in N Engl J. Med. 2004 Sep. 30; 351(14):1470]. New England Journal of Medicine 2004; 350: 2239-46.
4. Djavan B, Remzi M, Schulman C C, Marberger M, Zlotta A R. Repeat prostate biopsy: who, how and when? A review. Eur Urol 2002; 42: 93-103.
5. Epstein J I, Herawi M. Prostate needle biopsies containing prostatic intraepithelial neoplasia or atypical foci suspicious for carcinoma: implications for patient care. J Urol 2006; 175: 820-34.
6. Egevad L, Granfors T, Karlberg L, Bergh A, Stattin P. Prognostic value of the Gleason score in prostate cancer. BJU International 2002; 89: 538-42.
7. Pinthus J H, Witkos M, Fleshner N E, et al. Prostate cancers scored as Gleason 6 on prostate biopsy are frequently Gleason 7 tumors at radical prostatectomy: implication on outcome. Journal of Urology 2006; 176: 979-84.
8. Caprioli R M, Farmer T B, Gile J. Molecular imaging of biological samples: localization of peptides and proteins using MALDI-TOF MS. Anal Chem 1997; 69: 4751-60.
9. Chaurand P, Cornett D S, Caprioli R M. Molecular imaging of thin mammalian tissue sections by mass spectrometry. Curr Opin Biotechnol 2006; 17: 431-6.
10. Caldwell R L, Caprioli R M. Tissue profiling by mass spectrometry: a review of methodology and applications. Mol Cell Proteomics 2005; 4: 394-401.
11. Chaurand P, Sanders M E, Jensen R A, Caprioli R M. Proteomics in diagnostic pathology: profiling and imaging proteins directly in tissue sections. Am J Pathol 2004; 165: 1057-68.
12. Yanagisawa K, Shyr Y, Xu B J, et al. Proteomic patterns of tumour subsets in non-small-cell lung cancer. Lancet 2003; 362: 433-9.
13. Cornett D S, Mobley J A, Dias E C, et al. A novel histology-directed strategy for MALDI-MS tissue profiling that improves throughput and cellular specificity in human breast cancer. Molecular & Cellular Proteomics 2006; 5: 1975-83.
14. Reyzer M L, Caldwell R L, Dugger T C, et al. Early changes in protein expression detected by mass spectrometry predict tumor response to molecular therapeutics. Cancer Research 2004; 64: 9093-100.
15. Schwartz S A, Weil R J, Thompson R C, et al. Proteomic-based prognosis of brain tumor patients using direct-tissue matrix-assisted laser desorption ionization mass spectrometry. Cancer Res 2005; 65: 7674-81.
16. Lemaire R, Desmons A, Tabet J C, Day R, Salzet M, Fournier I. Direct Analysis and MALDI Imaging of Formalin-Fixed, Paraffin-Embedded Tissue Sections. J Proteome Res 2007; 6: 1295-305.
17. Schwamborn K, Krieg R C, Reska M, Jakse G, Knuechel R, Wellmann A. Identifying prostate carcinoma by MALDI-Imaging. International Journal of Molecular Medicine 2007; 20: 155-9.
18. Shimma S, Sugiura Y, Hayasaka T, Hoshikawa Y, Noda T, Setou M. MALDI-based imaging mass spectrometry revealed abnormal distribution of phospholipids in colon cancer liver metastasis. J Chromatogr B Analyt Technol Biomed Life Sci 2007.
19. Widmann C, Gibson S, Jarpe M B, Johnson G L. Mitogen-activated protein kinase: conservation of a three-kinase module from yeast to human. Physiol Rev 1999; 79: 143-80.
20. Su B, Cheng J, Yang J, Guo Z. MEKK2 is required for T-cell receptor signals in JNK activation and interleukin-2 gene expression. J Biol Chem 2001; 276: 14784-90.
21. Zhao Q and Lee F S. Mitogen-activated protein kinase/ERK kinase kinases 2 and 3 activate nuclear factor-kappaB through IkappB kinase-alpha and IkappaB kinase-beta. J. Biol. Chem. 1999; 274: 8655-61.
22. Nakamura K, Johnson G L. PB1 domains of MEKK2 and MEKK3 interact with the MEK5 PB1 domain for activation of the ERK5 pathway. Journal of Biological Chemistry 2003; 278:3 6989-92.
23. Uhlik M T, Abell A N, Cuevas B D, Nakamura K, Johnson G L. Wiring diagrams of MAPK regulation by MEKK1, 2, and 3. Biochemistry & Cell Biology 2004; 82: 658-63.
24. Nakamura K, Uhlik M T, Johnson N L, Hahn K M, Johnson G L. PB 1 domain-dependent signaling complex is required for extracellular signal-regulated kinase 5 activation. Molecular & Cellular Biology 2006; 26: 2065-79.
25. Van den Steen P E, Dubois B, Nelissen I, Rudd P M, Dwek R A, Opdenakker G. Biochemistry and molecular biology of gelatinase B or matrix metalloproteinase-9 (MMP-9). Crit. Rev Biochem Mol Biol 2002; 37: 375-536.
26. Mehta P B, Jenkins B L, McCarthy L, et al. MEK5 overexpression is associated with metastatic prostate cancer, and stimulates proliferation, MMP-9 expression and invasion. Oncogene 2003; 22: 1381-9.
27. McCracken S R, Ramsay A, Heer R, et al. Aberrant expression of extracellular signal-regulated kinase 5 in human prostate cancer. Oncogene 2008; 27: 2978-88.
28. Seyfried J, Wang X, Kharebava G, Tournier C. A novel mitogen-activated protein kinase docking site in the N terminus of MEK5alpha organizes the components of the extracellular signal-regulated kinase 5 signaling pathway. Molecular & Cellular Biology 2005; 25: 9820-8.
29. Goodwin R J, Dungworth J C, Cobb S R, Pitt A R. Time-dependent evolution of tissue markers by MALDI-MS imaging. Proteomics 2008; 8:3801-8.
30. Amann J M, Chaurand P, Gonzalez A, et al. Selective profiling of proteins in lung cancer cells from fine-needle aspirates by matrix-assisted laser desorption ionization time-of-flight mass spectrometry. Clinical Cancer Research 2006; 12:5142-50.
31. Schneider J J, Unholzer A, Schaller M, Schafer-Korting M, Korting H C. Human defensins. Journal of Molecular Medicine 2005; 83:587-95.
22. Fanger G R, Johnson N L, Johnson GL. MEK kinases are regulated by EGF and selectively interact with Rac/Cdc42. EMBO Journal 1997; 16:4961-72.

What is claimed is:

1. A method of diagnosing prostate cancer in a subject, comprising the steps of:
(a) obtaining one or more test samples from said subject;
(b) detecting the differential expression of at least one protein marker in the one or more test samples, wherein the protein marker is selected from:
M3373,
M3443,
M3488,
M4027,
M4274,
M4355
M4430,
M4635,
M4747,
M4972,
M8205, and
M10111; and
(c) correlating the detection of differential expression of at least one protein marker with a diagnosis of prostate cancer, wherein the correlation takes into account the amount of the at least one protein marker in the one or more test samples compared to a control amount of the at least one protein marker.

2. The method of claim 1 wherein one test sample is seminal plasma.

3. The method of claim 1 wherein one test sample is selected from the group consisting of blood, serum, urine, prostatic fluid, seminal fluid, semen, and prostate tissue.

4. The method of claim 1 wherein one or more test samples are first and second serial prostate tissue sections.

5. The method of claim 1 comprising detecting the differential expression at least one protein marker by gas phase ion spectrometry.

6. The method of claim 5 wherein gas phase ion spectrometry is laser desorption mass spectrometry.

7. The method of claim 6 wherein the laser desorption mass spectrometry is MALDI-IMS.

8. The method of claim 1 comprising detecting the differential expression of at least one protein marker by immunoassay.

9. The method of claim 1 comprising detecting a plurality of markers.

10. The method of claim 4 further comprising step (d) of staining the first serial prostate tissue section and comparing the stained first serial tissue section to the differential expression of at least one protein marker detected in the second serial prostate tissue section.

11. The method of claim 1 wherein at least one protein marker is MEKK2 or a fragment or variant thereof.

12. The method of claim 1 wherein said one or more test samples is obtained at biopsy or post-surgery.

13. A method of diagnosing prostate cancer in a subject, comprising the steps of:
(a) obtaining one or more test samples from said subject;
(b) detecting the differential expression of MEKK2, a fragment of MEKK2 or a variant of MEKK2 in the one or more test samples; and
(c) correlating the detection of differential expression of MEKK2, a fragment of MEKK2 or a variant of MEKK2 with a diagnosis of prostate cancer, wherein the correlation takes into account the amount of the MEKK2, fragment of MEKK2 or variant of MEKK2 in the one or more test samples compared to a control amount of the MEKK2, fragment of MEKK2 or variant of MEKK2.

14. A method of diagnosing metastatic prostate cancer in a subject, comprising the steps of:
(a) obtaining one or more test samples from prostate tumor tissue of said subject;
(b) detecting the differential expression of at least one protein marker in the one or more test samples, wherein the protein marker is selected from:
M4030,
M5364,
M9533,
M6186,
M3230,
M3817,
M3245,
M9767,
M8963,
M9091,
M9021, and
M6344; and
(c) correlating the detection of differential expression of at least one protein marker with a diagnosis of metastatic prostate cancer, wherein the correlation takes into account the amount of the at least one protein marker in the one or more test samples compared to a control amount of the at least one protein marker.

15. The method of claim 14 wherein one or more test samples are first and second serial prostate tumor tissue sections.

16. The method of claim 14 comprising detecting the differential expression at least one protein marker by gas phase ion spectrometry.

17. The method of claim 14 wherein gas phase ion spectrometry is laser desorption mass spectrometry.

18. The method of claim 17 wherein the laser desorption mass spectrometry is MALDI-IMS.

19. The method of claim 14 comprising detecting a plurality of markers.

20. The method of claim 14 further comprising step (d) of staining the first serial prostate tumor tissue section and comparing the stained first serial tumor tissue section to the differential expression of at least one protein marker detected in the second serial prostate tumor tissue section.

21. The method of claim 1 wherein said one or more test samples is obtained at biopsy or post-surgery.


SEQUENCE LISTING (9 sequences)

SEQ ID NO: 1 - protein, 36 aa, Homo sapiens
SLQETRKAKSSSPKKQNDVRVKFEHRGEKRILQFPR

SEQ ID NO: 2 - protein, 12 aa, Homo sapiens
DVRVKFEHRGEK

SEQ ID NO: 3 - protein, 18 aa, Homo sapiens
SSSPKKQNDVRVKFEHRG

SEQ ID NO: 4 - protein, 26 aa, Homo sapiens
KAKSSSPKKQNDVRVKFEHRGEKRIL

SEQ ID NO: 5 - protein, 6 aa, Homo sapiens
SLQETR

SEQ ID NO: 6 - protein, 5 aa, Homo sapiens
QNDVR

SEQ ID NO: 7 - protein, 4 aa, Homo sapiens
FEHR

SEQ ID NO: 8 - protein, 6 aa, Homo sapiens
ILQFPR

SEQ ID NO: 9 - protein, 100 aa, Homo sapiens
SMSLQETRKAKSSSPKKQNDVRVKFEHRGEKRILQFPRPVKLEDLRSKAKIAFGQSMDLHYTNNELVIPLTTQDDLDKAVELLDRSIHMKSLKILLVING